(19) United States
(12) Patent Application Publication
(10) Pub. No.: US 2025/0025383 A1
(43) Pub. Date: Jan. 23, 2025

(54) EMOLLIENT COMPOSITION IN EMULSION FORM

(71) Applicant: PIERRE FABRE MEDICAMENT, BOULOGNE-BILLANCOURT (FR)

(72) Inventors: Valérie MUGUET, TOULOUSE (FR); Jean-François CORDOLIANI, SAINTE FOY D'AIGREFEUILLE (FR)

(73) Assignee: PIERRE FABRE MEDICAMENT, BOULOGNE-BILLANCOURT (FR)

(21) Appl. No.: 18/909,622

(22) Filed: Oct. 8, 2024

Related U.S. Application Data
(62) Division of application No. 17/415,875, filed on Jun. 18, 2021.

(30) Foreign Application Priority Data
Dec. 21, 2018 (FR) 18 73902

(51) Int. Cl. A61K 8/06; A61K 8/31; A61K 8/34; A61K 8/73; A61K 8/81; A61K 8/86; A61K 8/891; A61Q 19/00

(52) U.S. Cl. CPC A61K 8/062; A61K 8/064; A61K 8/31; A61K 8/345; A61K 8/731; A61K 8/8158; A61K 8/86; A61K 8/891; A61Q 19/007; A61K 2800/30; A61K 2800/524; A61K 2800/594

(57) ABSTRACT

A novel paraben-free composition in oil in water or water in oil emulsion form includes water, glycerol, petrolatum, and liquid paraffin as well as at least one preservative different from parabens and at least two gelling agents of which one is a polyacrylamide type gelling agent. The composition is useful for the treatment of xerosis and/or cutaneous dryness states associated with dermatoses.

This application is a Divisional of co-pending application Ser. No. 17/415,875, filed on Jun. 18, 2021, which is the National Stage Application under 35 U.S.C. § 371 of International Application No. PCT/EP2019/086365, filed on Dec. 19, 2019, which claims the benefit under 35 U.S.C. § 119(a) to French Patent Application No. 18 73902, filed on Dec. 21, 2018, all of which are hereby expressly incorporated by reference into the present application.

The subject matter of the present invention is a novel composition in emulsion form comprising glycerol, Vaseline® petrolatum, and liquid paraffin as well as a particular system of preservatives and gelling agents.

Dryness of the skin is a common problem for a large part of the population. The possible origins of this condition are multiple and the particular manifestations variable, with symptoms such as scaling (dandruff), chapping, red blotches or itching. The dryness state of the skin may be transient and linked to particular behavioural or environmental conditions (for example use of cleaning products, treatment by certain medicines, climatic conditions, etc.), but may also be more chronic and associated with disruptions to the correct functioning of the skin, notably in certain pathological conditions such as xerosis or also atopic dermatitis, pruritis, ichthyosis, psoriasis, or other indications.

A key structure for moisturising the skin is formed by the corneal layer (stratum corneum), the uppermost layer of the epidermis. The corneal layer is composed of corneocytes, apoptotic cells which are the result of the ultimate phase of mutation of keratinocytes which progressively rise from the basal layer, and epidermal lipids, its lower part constitutes a real protective barrier against exogenous factors (pollution, sun, cold).

One of the primary functions of the corneal layer is notably to ensure the moisture content of the skin and to protect it against the loss of endogenous water. Various mechanisms may be cited in this context. The layer of corneocytes with their cornified protein envelope and the high concentration of lipids in the intercellular space represents a not very permeable barrier against the loss of moisture of the skin; a fundamental role is attributed notably to lipids of the ceramide family.

A large part of the water that is bound within the corneal layer is associated with the keratin fibres of corneocytes and with the hydrophilic parts of intercellular lipids. Corneocytes also contain a mixture designated “Natural Moisturizing Factor” (NMF), comprising amino acids, pyrrolidone carboxylic acid, lactic acid, urea, mineral ions, and other substances. These molecules, based on their hygroscopic and soluble properties, act as endogenous humectants and thus contribute to the fixation of water in the corneal layer and ensure good elasticity of the skin.

Conversely, a defect in the capacity of the stratum corneum to retain cutaneous moisture brings about effects of dryness of the skin. Disruptions to the homeostasis of moisturising manifest themselves at several levels. As example, changes in the ceramide content in the skin and in their structure could be linked to cutaneous xerosis; other factors in various pathophysiological states concern the structure of the lipid layer, the cohesion of corneocytes, the production of the natural moisturising factor (NMF) or also the operation of cell transporters of water and glycerol, aquaporins.

A mainstay of the treatment and/or the prevention of dryness of the skin is the use of moisturising products and emollients by topical route, with the aim of re-establishing and maintaining the moisturising of the skin and ensuring the correct appearance and elasticity (suppleness) thereof. The user finds at his disposal a certain number of these preparations, which may be useful at several levels, notably in the context of remoisturising of the skin, preventing the loss of humidity and in restoring and repairing the cutaneous barrier; particular preparations often have a combination of these functions. Consequently, these products are composed of different classes of ingredients such as occlusives, humectants, emollients and protein regenerators.

These preparations for topical application may be made available in emulsion form, with suitable ingredients which ensure the correct properties of the formulations for example on the level of stability, conservation, consistency, spreading, homogeneity, organoleptic quality, odour, feel, visual aspect, and/or other parameters. There still exists however a need to improve these compositions in order to adapt them better to the expectations of consumers, to new environmental or health risk considerations for certain ingredients, and to the updating of regulatory restrictions.

Indeed, several substances commonly used in dermatological and cosmetic preparations for topical application have recently been called into question. They are notably agents which are used to improve the conservation of compositions for cutaneous use, for example by protection against microbiological contamination. A group of products that is particularly controversial in the dermatological and cosmetic field are notably preservatives of the parabens class. Indeed, parabens are suspected of being carcinogenic and of disrupting the hormonal system. Formulators must thus find alternatives to the use of these molecules. Similar considerations apply to certain other preservatives, often associated with properties that are allergenic and/or irritant for the skin. Products such as butylated hydroxyanisole or BHA, phenoxyethanol, chlorocresol, benzoic acid and salts thereof (benzoates), benzyl alcohol, and formaldehyde donors may be cited.

Emulsions comprising an association of glycerol, Vaseline® petrolatum, and liquid paraffin such as described in the applications WO2009/138517 and WO2009/138515 are particularly useful compositions in the care of cutaneous dryness. However, these associations of glycerol, Vaseline® petrolatum, and liquid paraffin are combined with propyl parahydroxy benzoate or chlorocresol as preservatives. There thus exists a real need for innovative formulae which avoid these ingredients considered as problematic.

Yet, it proves to be difficult to replace said substances in question in a functionally equivalent manner while retaining the body of the formulation with the approved combination associating glycerol, Vaseline® petrolatum, and liquid paraffin and while maintaining the established organoleptic and physical/chemical properties. The choice of suitable preservatives is limited, and modification of the components leads to galenic problems which are difficult to overcome. This is particularly true when it is necessary to maintain constant the concentrations of the main ingredients of the formula, such as for example for the formula of an association of glycerol, Vaseline® petrolatum, and liquid paraffin at respective concentrations of around 15%, around 8% and around 2% by weight compared to the total weight of the formulation, and to do so moreover in the established conditions, notably in the case of a pH slightly above pH 7.

In terms of conservation, the application WO2016/008999 describes the conservation by high pressure of an emulsion for cutaneous application which contains among other things glycerol, white Vaseline® petrolatum and liquid paraffin, and is without addition of parabens or other preservatives. The conservation method is efficient at the microbiological level and from a physical/chemical viewpoint. However, the application of this technique requires the installation of quite an expensive apparatus and is not easy to carry out. In addition, the conservation and the rheological properties of the emulsion are not guaranteed for repeated use and/or use spread out over time, that is to say after opening the sterile vial.

There thus exists a real need for non-sterile, emollient, protective compositions for cutaneous use for all types of skin, including sensitive skin, comprising the association of glycerol, Vaseline® petrolatum, and liquid paraffin, exempt of irritant or allergenic preservatives and in particular parabens. Said emollient, protective composition must further have appropriate microbiological conservation, as defined for example by criteria A and/or B of the European Pharmacopoeia, 8^{th} Edition (2016), Chapter 5.1.3. Finally, this composition must have organoleptic properties and a consistency that is acceptable and stable over time. It is also expected that it is suited to repeated use and that the formulation is possible to produce at the industrial, technical and economic level.

In a surprising manner, the inventors of the present application have been able to develop particular compositions that meet all these criteria and more particularly long term microbiological, physical/chemical and rheological stability. The invention is all the more surprising in that it has been shown that the association of preservatives other than parabens in combination with a suitable system of gelling agents has made it possible to attain the desired result.

The present invention thus pertains to a composition in the form of an oil in water or water in oil emulsion, comprising
water at a concentration of between 30 and 80% by weight compared to the total weight of the composition,
glycerol at a concentration of between 10 and 20%, preferentially between 13 and 17%, by weight compared to the total weight of the composition,
Vaseline® petrolatum at a concentration of between 3 and 20%, preferentially between 5 and 10%, by weight compared to the total weight of the composition
liquid paraffin at a concentration of between 0.5 and 5%, further preferentially between 1 and 3%, by weight compared to the total weight of the composition,
at least one preservative different from parabens,
at least 2 gelling agents of which one polyacrylamide type gelling agent.

Preferred but non-limiting aspects of the composition according to the invention are the following: The composition according to the invention may be prepared in the form of a simple water in oil (W/O) or oil in water (O/W) emulsion, a multiple emulsion such as for example, a water in oil in water (W/O/W) emulsion or an oil in water in oil (O/W/O) emulsion, or further in the form of a hydrodispersion or a lipodispersion, a gel or an aerosol. Preferentially, the present invention is prepared in the form of an oil in water (O/W) emulsion.

Emulsions are intimate mixtures of two non-miscible liquid substances. They are always two liquids which in normal situation are non-miscible but which are going to, by specific operations (stirring, mixing, addition of emulsifiers), succeed in having a macroscopically homogenous but microscopically heterogenous aspect. Emulsions are composed of an oil phase, an aqueous phase and an appropriate emulsifying system.

In the aqueous phase of the emulsion, the composition according to the present invention contains water. Advantageously, water is comprised between 30 and 80% by weight compared to the total weight of the composition. Advantageously, the water used for the aqueous phase of the emulsion may be distilled water or thermal water having dermato-cosmetic properties.

The composition according to the invention contains glycerol (or 1,2,3-propanetriol). Advantageously, the glycerol has the criteria described and controlled according to the European Pharmacopoeia 8^{th} Edition (2016), Monograph n° 0496. The concentration of glycerol in the composition is comprised between 10 and 20%, preferentially between 13 and 17%, and in a particularly preferred manner is around 15% by weight compared to the total weight of the composition.

The composition according to the invention contains Vaseline® petrolatum (or petrolatum). Advantageously, the Vaseline® petrolatum has the criteria described and controlled according to the European Pharmacopoeia 8^{th} Edition (2016), Monograph n° 1799. The concentration of Vaseline® petrolatum is comprised between 3 and 20%, preferentially between 5 and 10% and in a particularly preferred manner is around 8% by weight compared to the total weight of the composition.

The composition according to the invention contains liquid paraffin (paraffinum perliquidum). Advantageously, the liquid paraffin has the criteria described and controlled according to the European Pharmacopoeia 8^{th} Edition (2016), Monograph no 0239. The concentration of liquid paraffin is comprised between 0.5 and 5%, preferentially between 1 and 3% and in a particularly preferred manner is around 2% by weight compared to the total weight of the composition.

In the composition according to the invention, the association of glycerol, Vaseline® petrolatum, liquid paraffin is present according to a proportion comprised between 10 and 50%, and preferentially between 20 and 30% by weight compared to the total weight of the composition. The concentration of glycerol is comprised between 10 and 20%, preferentially between 13 and 17%, and in a particularly preferred manner is around 15% by weight compared to the total weight of the composition. The concentration of Vaseline® petrolatum is comprised between 3 and 20%, preferentially between 5 and 10% and in a particularly preferred manner is around 8% by weight compared to the total weight of the composition. The concentration of liquid paraffin is comprised between 0.5 and 5%, preferentially between 1 and 3% and in a particularly preferred manner is around 2% by weight compared to the total weight of the composition.

Advantageously, the composition according to the invention comprises around 15% glycerol, around 8% Vaseline® petrolatum, around 2% liquid paraffin by weight compared to the total weight of the composition.

The composition according to the invention contains one or more preservatives, all different from parabens. The term “preservative” or “preservative agent” comprises any agent that is widely used to avoid the growth of microorganisms and/or to avoid degradation of the composition in question. Preferably, the composition according to the invention has between 0.01% and 20% of preservatives by weight compared to the total weight of the preparation, further preferentially between 0.1 and 15%.

The present invention is characterised in that it contains at least one preservative but does not contain parabens.

One subject matter of the invention relates to a composition for topical use which does not contain parabens. The expression “does not contain” parabens is taken to mean, in the sense of the present invention, that the composition is essentially devoid of parabens. The concentration of parabens will thus be able to be of the order of traces, that is to say a concentration of the order of 0%, particularly less than 0.01% by weight compared to the total weight of the preparation, further preferentially less than 0.003% or further less than 0.001% by weight compared to the total weight of the preparation. Such percentages reflect the character of absence, or of traces, of parabens in the formulation, knowing in effect that it is not possible to ensure an absolute and total absence due to the simple fact of possible, and minimum, contaminations inevitable during manufacture.

In the sense of the present invention, the term “paraben”, “parabens” or “paraben type preservative” refers to an ester of parahydroxybenzoic acid and to salts thereof. Notably, the term “parabens” refers to esters of parahydroxybenzoic acid as commonly used as preservatives. The term “parabens” thus corresponds to esters of parahydroxybenzoic acid, more particularly C1-C8 alkyl parahydroxybenzoates, that is to say an ester resulting from the condensation of parahydroxybenzoic acid with a C1-C8 alcohol and salts thereof (including the sodium and potassium salts), used alone or in combination.

The C1-C8 alkyl residue may be linear or branched, aliphatic or aromatic. It may be an alkyl residue selected from the group consisting in methyl, ethyl, propyl, isopropyl, butyl, isopropyl, pentyl, hexyl, octyl, benzyl.

As parabens may be cited esters of parahydroxybenzoic acid, such as methylparaben (or methyl 4-hydroxybenzoate), ethylparaben (or ethyl 4-hydroxybenzoate), propylparaben (or propyl 4-hydroxybenzoate), isopropylparaben, butylparaben, isobutylparaben, benzylparaben.

Parabens also comprise salts, in particular sodium and potassium salts.

In terms of parabens thus to avoid and of which it is sought to limit the presence or the concentration it is possible to cite in particular and in a non-exhaustive manner methylparaben or methyl 4-hydroxybenzoate (E218) and the sodium salt thereof (E219), ethylparaben or ethyl 4-hydroxybenzoate (E214) and the sodium salt thereof (E215), propylparaben or propyl 4-hydroxybenzoate (E216) and the sodium salt thereof (E217), butylparaben and the sodium salt thereof.

According to an embodiment of the present invention, it will also be sought to maintain the concentration of preservative selected from phenoxyethanol, butylated hydroxyanisole (or BHA), chlorocresol, and formaldehyde donors, used alone or in combination below a threshold of 0.01% by weight compared to the total weight of the preparation, further preferentially 0.003% or further 0.001% by weight compared to the total weight of the preparation. Further preferentially, the composition according to the present invention is also essentially devoid of, that is to say that it contains a concentration of the order of 0%, more particularly less than 0.01% by weight compared to the total weight of the preparation, further preferentially less than 0.003% or less than 0.001% by weight compared to the total weight of the preparation, of benzoic acid as well as salts thereof and/or benzyl alcohol.

In a preferential embodiment, the composition of the invention contains preservatives with the exclusion of those of paraben type, but also to the exclusion of phenoxyethanol, to the exclusion of butylated hydroxyanisole, with the exclusion of chlorocresol, with the exclusion of formaldehyde donors. The expression “with the exclusion of”, in the same way as the expression “does not contain”, signifies, in the sense of the present invention, that the composition is essentially devoid of the components in question. The concentration of said compounds will thus be able to be of the order of traces, that is to say a concentration of the order of 0%, particularly less than 0.01% by weight compared to the total weight of the preparation, further preferentially not more than 0.003% or not more than 0.001% by weight compared to the total weight of the preparation.

In a further preferential embodiment, the composition of the invention contains no preservative selected from parabens, benzoates, benzyl alcohol, phenoxyethanol, butylated hydroxyanisole, chlorocresol, alone or in combination, in particular none of these preservatives at a concentration of more than 0.01% by weight compared to the weight of the preparation, further preferentially not more than 0.003% or not more than 0.001% by weight compared to the total weight of the preparation.

In one of the embodiments thereof, the composition according to the present invention is also essentially devoid of formaldehyde donors, it thus contains between 0% and 0.01%, preferentially between 0% and 0.003%, further preferentially between 0% and 0.001% of formaldehyde donors by weight compared to the total weight of the preparation.

According to a particular embodiment, the composition according to the present invention is also essentially devoid of butylated hydroxytoluene or BHT, it thus contains between 0% and 0.01%, preferentially between 0% and 0.003%, further preferentially between 0% and 0.001% of butylated hydroxytoluene by weight compared to the total weight of the preparation.

In one of the embodiments thereof, the composition according to the present invention is essentially devoid of aromatic preservative, it thus contains between 0% and 0.01%, preferentially between 0% and 0.003%, further preferentially between 0% and 0.001% of aromatic preservative by weight compared to the total weight of the preparation.

The terms “essentially devoid of” as well as “essentially free”, “essentially exempt” and “essentially absent” in the sense of the present invention indicate that the substance concerned or the substances concerned are absent from the composition in question, or present as traces at a level as low as practically feasible; in particular at a concentration of the order of 0%, particularly less than 0.01% by weight compared to the total weight of the preparation, further preferentially less than 0.003% or less than 0.001% by weight compared to the total weight of the preparation. Further preferentially, the concentration of the substance concerned in the composition in question is below the limit of detection by applicable analysis methods normally used by those skilled in the art.

The compositions according to the present invention contain one or more preservatives, with the exclusion of parabens, to ensure the microbiological stability of the preparation. In view of the target population which includes children and infants, and adults with sensitive skin, the present invention aims to avoid or to limit irritant and/or allergenic preservatives. Another complementary manner of adapting to the conditions of an application on sensitive skin is to limit the concentration of the preservatives used. Certain preferential embodiments of the present invention contain combinations of two or several preservatives, particularly three or more preservatives, further more particularly 4 or more preservatives, with the exclusion of parabens, thus making it possible to meet the criteria in terms of efficiency of antimicrobial conservation and to limit at the same time the concentration of each of the preservatives thanks to their synergic interaction.

The at least one preservative, different from parabens, present in the composition according to the invention is selected from the group of suitable diols, in particular in the group comprising, or consisting in, hexanediol, ethylhexyl glycerin, pentylene glycol, butylene glycol, 1,2 octanediol (caprylyl glycol) as well as mixtures thereof.

According to an embodiment of the invention, the at least one preservative, different from parabens, present in the composition according to the invention is selected from the group of diols.

A suitable diol may be selected from the group comprising aliphatic alkane diols comprising 2 to 10 carbon atoms, particularly 4 to 8 carbon atoms, linear or branched.

Ethylhexyl glycerin is an ether of glycerol and is in this respect, in the context of the present invention, a suitable diol on account of the presence of two hydroxyl groups.

The at least one preservative, different from parabens, will be present at concentrations suited to ensuring the microbiological stability of the composition.

Advantageously, it contains one or more preservatives, different from parabens, selected from the group consisting in hexanediol, ethylhexyl glycerin, pentylene glycol, and mixtures thereof, at concentrations suited to ensuring the microbiological stability of the composition.

In an embodiment of the composition according to the invention, the composition according to the invention contains hexanediol. Hexanediol may be used alone or in combination with at least one other preservative. In a particular embodiment of the composition, it does not contain other preservative than hexanediol. When it is used as sole preservative, hexanediol is present in the composition according to the invention at a concentration of 3% to 10%, preferentially around 5% by weight compared to the total weight of the composition. In an embodiment of the composition according to the invention, hexanediol is used in combination with at least one other preservative, with a concentration of hexanediol of 0.2% to 3%, preferentially 0.3% to 2%, further preferentially around 0.4%, 0.45% or 0.5% by weight compared to the total weight of the composition.

In an embodiment thereof, the composition according to the invention contains ethylhexyl glycerin at a concentration of 0.15% to 1%, preferentially 0.2% to 0.6%, further preferentially around 0.3%, around 0.4% or around 0.5% by weight compared to the total weight of the composition. Ethylhexyl glycerin may be used alone or in combination with another preservative. In a preferential embodiment of the composition according to the invention, ethylhexyl glycerin is used in combination with at least one other preservative. In another embodiment of the composition, it does not contain other preservative than ethylhexyl glycerin.

According to a particular embodiment, the composition according to the invention contains pentylene glycol. Pentylene glycol may be used alone or in combination with at least one other preservative. In a particular embodiment of the composition, it does not contain other preservative than pentylene glycol. When it is used as sole preservative, pentylene glycol is present in the composition according to the invention at a concentration of 4% to 10%, preferentially around 5% by weight compared to the total weight of the composition. In another embodiment of the composition according to the invention, pentylene glycol is used in combination with at least one other preservative, with a concentration of pentylene glycol of 0.3% to 3%, preferentially 0.3% to 0.5%, further preferentially around 0.3%, around 0.4% or around 0.45% by weight compared to the total weight of the composition.

In an embodiment thereof, the composition according to the invention contains butylene glycol. Preferentially, butylene glycol is used in combination with at least one other preservative. Within an embodiment of the composition according to the invention, butylene glycol is used at a concentration of 1% to 10%, preferentially 2% to 5%, further preferentially at around 4% or around 4.4% by weight compared to the total weight of the composition.

In another embodiment of the composition according to the invention, it contains 1,2 octanediol (caprylyl glycol) at a concentration of 0.1% to 1%, preferentially around 0.3%, around 0.4% or around 0.5% by weight compared to the total weight of the composition. Preferentially, 1,2 octanediol (caprylyl glycol) is used in combination with at least one other preservative.

According to a particular embodiment, the composition according to the invention contains ethylhexyl glycerin at a concentration of 0.15% to 1%, preferentially 0.2% to 0.6%, further preferentially around 0.3%, around 0.4% or around 0.5% by weight compared to the total weight of the composition and pentylene glycol at a concentration of 0.1% to 3%, preferentially 0.3% to 0.5%, further preferentially around 0.3%, around 0.4% or around 0.45% by weight compared to the total weight of the composition, and, further advantageously, it does not contain other preservative than ethylhexyl glycerin and pentylene glycol. In such a composition, said preservatives will be able to be advantageously associated with a mixture of two gelling agents, said mixture of gelling agents comprising, or consisting in, a first gelling agent of polyacrylamide type being the copolymer of acrylamide/sodium acryloyldimethyltaurate with isohexadecane and polysorbate in quantity of around 0.5%, or around 0.6%, by weight compared to the total weight of the composition; a second gelling agent being a carbomer, in particular Carbomer® 980, in quantity of around 0.025%, or around 0.05%, by weight compared to the total weight of the composition.

In an embodiment thereof, the composition according to the invention contains ethylhexyl glycerin at a concentration of 0.15% to 1%, preferentially 0.2% to 0.5%, further preferentially 0.3% to 0.5% by weight compared to the total weight of the composition, and butylene glycol at a concentration of 1% to 10%, preferentially 2% to 4.4% by weight compared to the total weight of the composition, and, further advantageously, it does not contain other preservative than ethylhexyl glycerin and butylene glycol.

In another embodiment thereof, the composition according to the invention contains ethylhexyl glycerin at a concentration of 0.15% to 1%, preferentially 0.2% to 0.5%, further preferentially 0.3% to 0.5% by weight compared to the total weight of the composition, and 1,2 octanediol (caprylyl glycol) at a concentration of 0.1% to 1%, preferentially 0.3% by weight compared to the total weight of the composition, and, further advantageously, it does not contain other preservative than ethylhexyl glycerin and 1,2 octanediol (caprylyl glycol).

The composition according to the invention contains at least two gelling agents comprising a polyacrylamide type gelling agent. The polyacrylamide may be a homopolymer or a copolymer of acrylamide with other monomers. This other monomer may in particular be acryloyldimethyltaurate, (meth)acrylic acid, esters of (meth)acrylic acid and mixtures thereof. In particular the polyacrylamide may be an acrylamide/acryloyldimethyltaurate copolymer. The comonomer may be in acid form or neutralised with an alkaline or alkaline-earth agent.

Appropriate gelling agents contain (in a non-limiting manner) stabilising polymers such as xanthan gum, gellan gum (E4183), carbomers (synthetic polymers of acrylic acid) such as for example Carbomer® 974 or Carbomer® 980, hydroxyethyl cellulose (for example Hypromellose 2910), hydroxypropyl methylcellulose (HPMC), carboxymethyl cellulose. In particular the at least two gelling agents of the composition according to the invention comprise a gelling agent of polyacrylamide polymer type. As gelling agent of the polyacrylamide family, the copolymer mixture of acrylamide/sodium acryloyldimethyltaurate/isohexadecane/polysorbate 80 sold by the SEPPIC Company, the mixture polyacrylamide/isoparaffin C13-14/laureth-7 sold by the SEPPIC Company (as an example, the products of the Seppic Company of the Sepineo®, Sepigel® or Simulgel® range) may be cited. The copolymer mixture of acrylamide/sodium acryloyldimethyltaurate with isohexadecane and polysorbate-(also called copolymer mixture of acrylamide/sodium acryloyldimethyltaurate or further copolymer acrylate/sodium acryloydimethyl taurate & Isohexadecane & Polysorbate 80) will be designated in the examples by “acrylamide copolymer mixture”. As non-limiting examples of gelling agents may be cited the carbomers sold under the name Ultrez 20®, Ultrez 10®, Carbopol 1382® or Carbopol ETD2020NF®, Carbopol 981 or further Carbopol 980 by the Lubrizol Company, polysaccharides with as non-limiting examples xanthan gum such as Xanturall80® sold by the Kelco Company, gellan gum sold under the name Kelcogel by the Kelco Company, guar gum, cellulose and derivatives thereof such as microcrystalline cellulose and sodium carboxymethyl cellulose sold under the name Avicel CL-611 by the FMC Biopolymer Company, hydroxypropyl methylcellulose in particular the product sold under the name Methocel E4M premium by the Dow Chemical Company or hydroxyethyl cellulose, in particular, the product sold under the name Natrosol HHX 250® by the Ashland Company, sodium carboxymethyl cellulose, in particular Blanose 7F cellulose gum sold by the Ashland Company, the family of aluminium magnesium silicates such as Veegum K sold by the Vanderbilt Company, the family of modified starches such as modified potato starch sold under the name Structure Solanace or instead mixtures thereof, the family of carrageenans in particular categorised into four major families: κ, λ, β, ω such as Viscarin® and Gelcarin® marketed by the IMCD Company and mixtures thereof.

Carbomers are synthetic hydrophilic polymers of acrylic acid, of high molecular weight. Carbomers are produced by polymerisation of acrylic acid to form crosslinked polymers of acrylic acid (polyacrylic acid), of high molecular weight, then optionally crosslinked for example with ethers of pentaerythritol. They may be homopolymers of acrylic acid, linear or crosslinked with a crosslinking agent such as an allylether of pentaerythritol, an allylether of saccharose or an allylether of propylene (the products Carbopol® of Lubrizol may be cited for example).

Carbomer is the generic name of a class of molecules absorbing water in very high quantities. Carbomers are crosslinked polymers of acrylic acid of high molecular weight which, once neutralised, have the capacity to absorb and retain water, which gives a gel. Most carbomers are classed as having a long or short rheology to indicate the nature of the polymer and the level of crosslinking. A short rheology corresponds to a highly crosslinked polymer, whereas a long rheology corresponds to a slightly crosslinked polymer.

In the case of the present invention crosslinked carbomers, in particular highly crosslinked carbomers, will be preferred.

Carbomer® 980 or Carbomer® 974 will in particular be preferred.

In an embodiment, the composition according to the invention contains between 0.1 and 0.4% of xanthan gum, preferably around 0.1% by weight compared to the total weight of the composition or 0.2% by weight compared to the total weight of the composition.

Advantageously, the composition according to the invention has between 0.2 and 2% of gelling mixture of polyacrylamide type, preferably between 0.3% and 1%, and further preferentially around 0.5% by weight compared to the total weight of the composition. In another embodiment thereof, it comprises around 0.6% of gelling mixture of polyacrylamide type by weight compared to the total weight of the composition. In yet another embodiment thereof, it comprises around 0.75% of gelling mixture of polyacrylamide type by weight compared to the total weight of the composition.

Further advantageously, the gelling mixture of polyacrylamide type of the composition is the copolymer mixture of acrylamide/sodium acryloyldimethyltaurate with isohexadecane and polysorbate. Preferentially, the composition according to the invention thus has between 0.2 and 2% of the copolymer mixture of acrylamide/sodium acryloyldimethyltaurate with isohexadecane and polysorbate, preferably between 0.5% and 1%, and further preferentially around 0.5% by weight compared to the total weight of the composition. In another embodiment, it comprises around 0.6% of copolymer mixture of acrylamide/sodium acryloyldimethyltaurate with isohexadecane and polysorbate by weight compared to the total weight of the composition. In yet another embodiment, it comprises around 0.75% of copolymer mixture of acrylamide/sodium acryloyldimethyltaurate with isohexadecane and polysorbate by weight compared to the total weight of the composition.

Advantageously, the composition according to the present invention contains a polyacrylamide type gelling agent in combination with one or more other gelling agents. Preferentially, said polyacrylamide type gelling agent is a copolymer mixture of acrylamide/sodium acryloyldimethyltaurate with isohexadecane and polysorbate.

In a preferential embodiment, the composition according to the invention has between 0.2 and 2% of polyacrylamide type gelling agent, advantageously the copolymer mixture of acrylamide/sodium acryloyldimethyltaurate with isohexadecane and polysorbate, preferably between 0.5% and 1%, and further preferentially around 0.5% or 0.6% by weight compared to the total weight of the composition in combination with at least one other gelling agent. Preferentially, the other gelling agent is selected from the group comprising xanthan gum, carbomers, hydroxyethyl cellulose (Hypromellose 2910), hydroxypropyl methyl cellulose (HPMC), used alone or in a mixture. Further preferentially, the other gelling agent is a carbomer, for example Carbomer® 980.

Advantageously, the composition according to the invention has between 0.01 and 0.5% of Carbomer® 980, preferably between 0.025% and 0.1%, preferably around 0.025%, around 0.03% or around 0.05% by weight compared to the total weight of the composition.

According to a preferential embodiment, the composition according to the present invention contains both the copolymer mixture of acrylamide/sodium acryloyldimethyltaurate with isohexadecane and polysorbate; and a carbomer, in particular Carbomer® 980, with a concentration of copolymer mixture of acrylamide/sodium acryloyldimethyltaurate with isohexadecane and polysorbate preferably between 0.5% and 1%, and further preferentially around 0.5% by weight compared to the total weight of the composition or around 0.6% by weight compared to the total weight of the composition; and a concentration of carbomer, in particular Carbomer® 980, preferably between 0.025% and 0.1%, further preferentially around 0.025%, around 0.03% or around 0.05% by weight compared to the total weight of the composition. In a preferred manner, the composition according to the invention comprises two preservatives, other than parabens, consisting in ethylhexyl glycerin and pentylene glycol; in association with a mixture of two gelling agents, said mixture of gelling agents comprising or consisting in:
a polyacrylamide copolymer type gelling agent being acrylamide/sodium acryloyldimethyltaurate with isohexadecane and polysorbate in quantity of around 0.2 to 1%, particularly 0.3 to 0.7% by weight compared to the total weight of the composition;
a second gelling agent being a carbomer, in particular Carbomer® 980, in quantity of around 0.01 to 0.1%, particularly between 0.02 to 0.05% by weight compared to the total weight of the composition.

The concentration of gelling agents is chosen as a function of the consistency of the final composition. This target consistency may be measured, at room temperature, on the date of manufacture (T0) and/or after storage for 3 months or 6 months, or even 12 months. In a particular embodiment, the consistency may be measured, at room temperature, after storage of the composition at 40° C. and 25% relative humidity for 3 months or 6 months.

Advantageously, the concentration of the gelling agent or gelling agents is thus chosen in such a way that makes it possible to obtain a composition according to the invention of a consistency at manufacture (T0) comprised between 70 and 230 g at room temperature and atmospheric pressure of 760 mm Hg, in a preferred manner between 80 and 220 g and in a more preferred manner between 90 and 210 g.

The measurement of consistency is given for a room temperature of around 20° C. and under an atmospheric pressure of around 760 mm Hg. The consistency is measured according to modalities well known to those skilled in the art. Several ways may be cited.

The measurement of consistency is a texturometric or penetrometric measurement of a product of a substance, according to which a sample of said product of said substance is placed in a support, a calibrated probe is made to penetrate from the top to the bottom of this sample, according to a course to travel in a volume of the mass of said sample and according to a speed chosen beforehand. Known devices, suited to this measurement method, called consistometry or texturometry or penetrometry texture analysers have as common principle of making a probe, with a determined calibre and/or weight, for example a cylinder or an inverted cone, penetrate into a volume of the mass of the sample. These systems may be based on the penetration depth reached in a given time, or on the time taken by the probe to cover a certain distance, or further on the force, in grammes, to exert so that the probe covers a certain distance in a certain time, at a determined temperature. In commercially available texture penetrometers, a motor makes a probe, fixed to a support moveable in vertical translation, descend into the sample, which is placed on a fixed platform. A force sensor integral with the probe and/or its moveable support measures the reaction of the sample to the penetration of the probe, that is to say a reaction force directed upwards and expressed in grammes.

In another embodiment of the invention, the concentration of the gelling agent or gelling agents is chosen in such a way that it makes it possible to obtain a composition according to the invention with a consistency value comprised between 40 and 210 g at room temperature, preferably between 50 and 200 g, further advantageously between 60 and 190 g when this consistency value is determined after storage at 40° C. and 25% relative humidity for 3 months, also further preferentially after storage for 6 months.

Further advantageously, the concentration of gelling agent or gelling agents is chosen in such a way that it makes it possible to obtain a composition according to the invention of a consistency comprised between 70 and 230 g at room temperature, in a preferred manner between 80 and 220 g and in a more preferred manner between 90 and 210 g at manufacture, and a consistency comprised between 40 and 210 g, preferably between 50 and 200 g, further advantageously between 60 and 190 g after storage at 40° C. and 25% relative humidity for 3 months, also further preferentially after storage for 6 months.

According to a preferential embodiment, the composition according to the present invention contains both a mixture of polyacrylamide type gelling agent, advantageously the copolymer mixture of acrylamide/sodium acryloyldimethyltaurate with isohexadecane and polysorbate; and a second gelling agent, Carbomer® 980. The concentration of polyacrylamide type gelling mixture is preferably between 0.5% and 1%, and further preferentially around 0.5% by weight compared to the total weight of the composition or around 0.6% by weight compared to the total weight of the composition. The concentration of Carbomer® 980 is preferably between 0.01 and 0.1%, particularly between 0.025% and 0.1%, further preferentially around 0.025%, around 0.03% or around 0.05% by weight compared to the total weight of the composition, and the consistency value of the composition at manufacture is comprised between 70 and 230 g at room temperature, in a preferred manner between 80 and 220 g and in a more preferred manner between 90 and 210 g.

According to another preferential embodiment, the composition according to the present invention contains both the copolymer mixture of acrylamide/sodium acryloyldimethyltaurate with isohexadecane and polysorbate; and a second gelling agent, Carbomer® 980. The concentration of the copolymer mixture of acrylamide/sodium acryloyldimethyltaurate with isohexadecane and polysorbate is preferably between 0.2% and 1%, and further preferentially between 0.3 and 0.7%, more particularly around 0.5% by weight compared to the total weight of the composition or further around 0.6% by weight compared to the total weight of the composition. The concentration of Carbomer® 980 is preferably between 0.01% and 0.1%, in particular between 0.02 and 0.05%, further preferentially around 0.025%, around 0.03% or around 0.05% by weight compared to the total weight of the composition, and the consistency value of the composition after storage at 40° C. and 25% relative humidity for 3 months is comprised between 40 and 210 g at room temperature, preferably between 50 and 200 g, further advantageously between 60 and 190 g; further preferentially, these consistency values are also maintained after storage for 6 months at 40° C. and 25% relative humidity.

Further preferentially, the composition according to the present invention contains both the copolymer mixture of acrylamide/sodium acryloyldimethyltaurate with isohexadecane and polysorbate, and the second gelling agent, Carbomer® 980. The concentration of copolymer mixture of acrylamide/sodium acryloyldimethyltaurate with isohexadecane and polysorbate is preferably between 0.5% and 1%, and further preferentially around 0.5% by weight compared to the total weight of the composition or around 0.6% by weight compared to the total weight of the composition. The concentration of Carbomer® 980 is preferably between 0.025% and 0.1%, further preferentially around 0.025%, around 0.03% or around 0.05% by weight compared to the total weight of the composition, and the consistency value of the composition at manufacture is comprised between 70 and 230 g at room temperature, in a preferred manner between 80 and 220 g and in a more preferred manner between 90 and 210 g, the consistency value of the composition after storage at 40° C. and 25% relative humidity for 3 months is comprised between 40 and 210 g at room temperature, preferably between 50 and 200 g, further advantageously between 60 and 190 g, and, optionally, the consistency value of the composition after storage at 40° C. and 25% relative humidity for 6 months is also comprised between 40 and 210 g at room temperature, preferably between 50 and 200 g, further advantageously between 60 and 190 g.

Advantageously, the composition according to the invention comprises between 10 and 20%, preferentially between 13 and 17%, and in a particularly preferred manner around 15% of glycerol, between 3 and 20%, preferentially between 5 and 10% and in a particularly preferred manner around 8% of Vaseline® petrolatum, between 0.5 and 5%, preferentially between 1 and 3% and in a particularly preferred manner around 2% of liquid paraffin by weight compared to the total weight of the composition, ethylhexyl glycerin at a concentration of 0.15% to 1%, preferentially 0.2% to 0.5%, further preferentially around 0.3%, around 0.4% or around 0.5% by weight compared to the total weight of the composition, pentylene glycol at a concentration of 0.3% to 3%, preferentially 0.3% to 0.45%, further preferentially around 0.3%, around 0.4% or around 0.45% by weight compared to the total weight of the composition, and, further advantageously, it does not contain other preservative than ethylhexyl glycerin and pentylene glycol, in particular no parabens. Preferably, said composition contains at the same time both the copolymer mixture of acrylamide/sodium acryloyldimethyltaurate with isohexadecane and polysorbate in association with Carbomer® 980. With a concentration of copolymer mixture of acrylamide/sodium acryloyldimethyltaurate with isohexadecane and polysorbate preferably between 0.5% and 1%, and further preferentially around 0.5% by weight compared to the total weight of the composition or around 0.6% by weight compared to the total weight of the composition, a concentration of carbomer, in particular Carbomer® 980, preferably between 0.025% and 0.1%, further preferentially around 0.025%, around 0.03% or around 0.05% by weight compared to the total weight of the composition.

The composition according to the invention comprises, further, the usual dermatologically and/or cosmetically compatible ingredients. A dermatologically and/or cosmetically compatible ingredient may be any ingredient among those known to those skilled in the art with a view to obtaining a composition for topical application in the form of a cream, a lotion, a gel, an ointment, an emulsion, a microemulsion, a spray, etc.

Thus, the composition according to the invention may further contain additives and formulation aids, such as emulsifiers, thickeners, water scavengers, spreading agents, stabilisers. Those skilled in the art will adapt the choice of these additives as a function of the expected effect.

Thus, the composition according to the invention may further comprise one or more compounds selected from the group constituted of fragrances, colorants, vitamins, pH correctors.

According to a preferential embodiment, the composition according to the present invention does not contain fragrance.

Appropriate emulsifiers comprise stearic acid, trolamine, PEG-40-stearate.

Advantageously, the composition according to the invention comprises one or more emulsifiers.

Advantageously, the composition according to the invention has between 1 and 5% of stearic acid, preferably around 3% by weight compared to the total weight of the composition.

Advantageously, the composition according to the invention has between 0.2 and 2% of trolamine, preferably around 0.5% by weight compared to the total weight of the composition.

Advantageously, the composition according to the invention has between 0 and 2% of PEG-40-stearate, preferably around 0.5% by weight compared to the total weight of the composition.

Appropriate thickeners comprise glycerol monostearate, PEG (polyethylene glycol), in particular PEG 600.

Advantageously, the composition according to the invention comprises one or more thickeners.

Advantageously, the composition according to the invention has between 2 and 10% of glycerol monostearate, preferably around 5% by weight compared to the total weight of the composition.

Advantageously, the composition according to the invention has between 2 and 10% of polyethylene glycol, preferably around 5% by weight compared to the total weight of the composition. Preferentially, it is polyethylene glycol 600.

Advantageously, the composition according to the invention has between 2 and 10% of PEG 600, preferably around 5% by weight compared to the total weight of the composition.

Preferably, the composition according to the invention contains one or more spreading agents. Appropriate spreading agents comprise dimethicone, polydimethyl cyclosiloxane, isopropyl myristate, isopropyl palmitate, cetostearyl isononanoate, decyl oleate, ethyl oleate.

Advantageously, the composition according to the invention has between 0.2 and 2% of dimethicone, preferably around 0.5% of dimethicone by weight compared to the total weight of the composition.

In an embodiment of the invention, the composition according to the invention has between 1 and 3% of polydimethyl cyclosiloxane, preferably around 2.5% by weight compared to the total weight of the composition. In another invention embodiment, the composition according to the invention contains less than 0.1% of polydimethyl cyclosiloxane, advantageously it does not contain polydimethyl cyclosiloxane.

Advantageously, the composition according to the invention has between 0.5 and 2.5% of isopropyl myristate, preferably around 1% by weight compared to the total weight of the composition.

Advantageously, the composition according to the invention has between 0.5 and 2.5% of isopropyl palmitate, preferably around 1% by weight compared to the total weight of the composition.

Advantageously, the composition according to the invention is in the form of a white emulsion. Preferentially, it does not contain colorant, which is understood that there is no colorant which is added during the manufacture of the composition. Advantageously, the composition according to the invention has a suitable odour, preferably with a neutral evocation. Preferentially, it does not contain fragrance, which means that there is no fragrance added during manufacture of the composition.

From a comfortable use point of view, the emulsion for topical use in the sense of the present invention must have a pleasant texture, a homogenous aspect and an appropriate odour.

In terms of physical/chemical criteria, notably a good consistency and viscosity of the preparation are noted and the particle size (the size of the globules) as well as a pH range established for the cutaneous use of the emulsions in question. Those skilled in the art know established methods for the quantification of these parameters.

Advantageously, the composition according to the invention has a slightly basic pH (measured to the 1/10 in water), with a value comprised between pH 7.1 and pH 8.9, preferentially between pH 7.4 and pH 8.7.

Advantageously, at least 95% of the globules within the preparation have a size less than 30 μm, further advantageously less than 25 μm.

The spreadability is approached by consistency measurements, conducted by means of a texture analyser, it is expressed in g, at room temperature. The sought after consistency must enable good spreading of the product on the skin and must thus be sufficiently high at manufacture (T0) to ensure a suitable consistency value throughout the lifetime of the product. An acceptable expiry date value is around 40 g at room temperature, value below which the emulsion becomes too fluid to have acceptable prehension (and film forming power).

The consistency of the composition according to the invention at manufacture (T0) is preferably comprised between 70 and 230 g at room temperature, in a preferred manner between 80 and 220 g and in a more preferred manner between 90 and 210 g. Given a certain possible (and tolerable) drop in the consistency of a cream over time, as well known to those skilled in the art, the consistency of the composition after storage for 3 months at room temperature is preferably comprised between 40 and 210 g at room temperature, in a preferred manner between 50 and 200 g and in a more preferred manner between 60 and 190 g. Further preferentially, these values are also measured after storage at 40° C. and 25% relative humidity for 6 months.

The viscosity of the composition according to the invention at manufacture is preferably comprised between 7000 mPa·s and 40000 mPa·s at room temperature, in a preferred manner between 9000 mPa·s and 30000 mPa·s. After storage for 3 months at 40° C. and 25% relative humidity the viscosity value is preferably comprised between 6000 mPa·s and 30000 mPa·s at room temperature, in a preferred manner between 7000 mPa·s and 25000 mPa·s. Further preferentially, these values are also measured after storage at 6 months at 40° C. and 25% relative humidity.

The measurement of viscosity consists in measuring the shear parameters of the material in rotating spindle rheometers or viscosimeters. The sample is placed in a cup forming the stator, in which is engaged a rotor which may have various shapes depending on the type of apparatus. The rotor is moved by means of a motor; the greater the viscosity, the more the rotor is braked. The resistance value caused by the viscosity of the material situated in the air gap between rotor and stator is read on a viscosity scale.

In a particular aspect of the invention, the compositions according to the invention are stable over time. The parameters established for the composition according to the invention must be maintained over time, in the sense that a degradation of the preparation is to be avoided.

Criteria with respect to the microbiological stability of preparations for topical use may be consulted in the European Pharmacopoeia, with values defined for several types of microorganisms, notably bacteria as well as yeasts and fungi. Microbiological criteria are for example defined in the European Pharmacopoeia 8^{th} Edition (2016) § 5.1.3 for topical products applicable by cutaneous route.

The stability of the compositions according to the invention may also be evaluated by the maintaining of the visual aspect as assessed by inspection after storage under conditions as defined; a dephasing of the product and a precipitation of the ingredients are to be avoided. In another aspect, this stability also concerns the absence of significant alterations of the olfactive characteristics over time, evaluated by olfactive sensation after storage under the conditions as defined.

A desired effect in a preferential embodiment of the invention is obtaining an emulsion stable to heat during storage. A degradation of the organoleptic characters of the preparation is thus to be avoided, even under condition of storage at high temperature. Advantageously, the composition according to the invention does not exhibit dephasing (assessed by inspection) 3 or 6 months after manufacture, or even 8 or 12 months after manufacture, preferentially even under conditions of storage at elevated temperature, for example at 40° C. or even at 50° C. Similarly, a precipitation of the compounds (assessed by inspection) of the emulsion is to be avoided, also preferentially under conditions of storage at 40° C. or 50° C. for 3 or 6 months, or even 8 or 12 months.

Even if a certain variability over time, notably in the parameters of consistency and viscosity of a pasty emulsion, is well known and acceptable, a too significant alteration of the consistency and the viscosity for the storage of these compositions is to be avoided.

According to an embodiment of the present invention, the composition according to the invention is stable over time. The expression “stable over time” is taken to mean that the consistency values, at room temperature, after storage do not vary in too significant a manner compared to the values at manufacture (T0); preferentially, the consistency value, at room temperature, of a composition after storage at 40° C. and 25% relative humidity for 3 months, or even 6 months, does not differ by more than 50%, further preferentially not more than 40%, and further more particularly not more than 30%, compared to the consistency value at room temperature of said composition at T0. Preferentially, the composition according to the invention has however a consistency value comprised between 40 and 210 g at room temperature, in a preferred manner between 50 and 200 g and in a more preferred manner between 60 and 190 g after storage at 40° C. and 25% relative humidity for 3 months, or even 6 months.

In another embodiment of the present invention, the viscosity values after storage must not vary in a too significant manner compared to the values at manufacture (T0); also the expression “stable over time”, is taken to mean that, preferentially, the viscosity value, at room temperature, of a composition after storage at 40° C. and 25% relative humidity for 3 months, or even 6 months, does not differ by more than 50%, particularly not more than 40%, compared to the composition at T0. Preferentially, the composition according to the invention has however a viscosity value comprised between 6000 mPa·s and 30000 mPa·s at room temperature, in a preferred manner between 7000 mPa·s and 25000 mPa·s, after storage at 40° C. and 25% relative humidity for 3 months, or even 6 months.

The expression “stable over time” signifies that the consistency does not vary as indicated above.

The expression “stable over time” signifies that the viscosity does not vary as indicated above.

The expression “stable over time” signifies that the consistency or the viscosity do not vary as indicated above.

The expression “stable over time” signifies that the consistency and the viscosity do not vary as indicated above.

The composition according to the invention may be a pharmaceutical composition or a cosmetic composition. It is intended for topical use.

The composition according to the invention is considered as an emollient.

An emollient according to the invention is a composition having the properties of softening and of relaxing the tissues of the body, in this instance the skin.

The subject matter of the present invention is also the composition according to the invention for the use thereof in the treatment of dry skin, cutaneous dryness states, in particular in infants or the elderly.

The composition according to the invention is useful for any xerosis indication and for any population for which an emollient may be used.

The subject matter of the present invention is the use of the composition according to the invention as emollient.

The composition according to the present invention is particularly suited to paediatrics and notably infants.

The composition according to the present invention is also particularly suited to the elderly, notably for senile or asteatosis xerosis.

The subject matter of the present invention is also the cosmetic use of the composition according to the invention, in particular in infants or the elderly, preferably for preventing cutaneous dryness states.

The subject matter of the present invention is also the composition according to the invention for the use thereof in the prevention or the treatment of iatrogenic xeroses and other secondary effects of treatments requiring the application of an emollient.

According to another embodiment, the subject matter of the present invention is also a composition according to the invention for the use thereof in the prevention or the treatment of the signs and symptoms of cutaneous dryness states (xerosis), notably within the context of certain dermatoses (dermatitis).

The subject matter of the present invention is also a composition according to the invention for the use thereof in the prevention or the treatment of superficial burns of small areas.

The subject matter of the present invention is also a composition according to the invention for the use thereof in the prevention or the treatment of eczema outbreaks observed in patients suffering from atopic dermatitis.

The invention thus relates to a composition according to the invention, for the use thereof as medical device.

The invention further relates to a composition according to the invention, for the use thereof in the prevention and/or the treatment of iatrogenic xeroses and other secondary effects of treatments requiring the application of an emollient.

The invention also relates to a composition according to the invention, for the use thereof in the prevention and/or the treatment of xeroses appearing as secondary effects or cutaneous symptoms of pathologies such as renal or diabetic insufficiency.

It is an aim to provide a composition according to the invention for the use thereof in the prevention and/or the treatment of cutaneous dryness states associated with certain dermatoses such as atopic dermatitis, ichthyosis states, psoriasis.

It is an aim to provide a composition according to the invention for the use thereof to decrease the frequency and/or reduce the intensity of eczema outbreaks observed in patients suffering from atopic dermatitis.

It is an aim to provide a composition according to the invention for the use thereof in the prevention and/or the treatment of superficial burns.

It is an aim to target a cosmetic use of a composition according to the invention, in particular in infants or the elderly.

It is an aim to target a cosmetic use of a composition according to the invention for preventing cutaneous dryness states.

### EXPERIMENTAL PART

the following examples illustrate the invention without limiting the scope thereof.

### Example 1) Screening of Preservatives

Firstly, a screening of preservatives is carried out. The aim is to determine if the preservatives that are included in the formula ensure protection against microbiological contamination. The criteria applied for this evaluation are those of the European Pharmacopoeia, 8^{th} Edition (2016), Paragraph § 5.1.3, as detailed hereafter. A first evaluation of the compatibility of the preservatives in view of other aspects (for example odour, aspect, potential irritant or allergenic) is also carried out.

All the preservatives are tested in a formulation of the following composition:
15% of glycerol,
8% of Vaseline® petrolatum,
2% of liquid paraffin
between 3% and 4% of emulsifiers (3% stearic acid+<1% TEA or TRIS)
5% of glycerol monostearate,
5% of polyethylene glycol,
spreading agents,
made up to 100% with purified water.

Incorporation of preservatives while hot, in the aqueous phase or in the oil phase, depending on the solubilities of the preservatives.

### Microbiological Test:

The formulations are voluntarily contaminated by around 106 germs/mL among the strains Pseudomonas aeruginosa (PA), Staphylococcus aureus (SA), Escherichia coli (EC), Candida albicans (CA), Aspergillus brasiliensis (AB), selected individually or used in a mixture.

The monitoring of the microbiological quality of the samples is carried out by counting microorganisms at different times: one day after treatment (D1), 7 days after treatment (D7), 14 days after treatment (D14), 28 days after treatment (D28).

Each time, the aptitude to preserve the composition against microbiological contamination is assessed by the reduction in the number of microorganisms, expressed in log (or, at 28 days, by the absence of increase). The criteria A and B of the European Pharmacopoeia, 8^{th} Edition (2016), Paragraph § 5.1.3 for topical products applicable by cutaneous route are the following:

TABLE 1

| Reduction (log) | | 2 days | 7 days | 14 days | 28 days |
|---|---|---|---|---|---|
| Bacteria | Criterion A | 2 log | 3 log | | No |
| | Criterion B | — | — | 3 log | increase |
| Yeasts | Criterion A | — | — | 2 log | |
| and | Criterion B | — | — | 1 log | |
| moulds | | | | | |

Given the number of tests, only the compliance or non-compliance of the tested compositions is indicated without detail of the values of reduction for the different strains.

TABLE 2

| | | Results of | |
|---|---|---|---|
| | | microbiological | |
| | | tests | |
| | Concentrations | (compliance | Comments/ |
| Preservative tested | tested | with criteria) | other aspects |
| Formula without | (control) | neither criteria | |
| preservative | | A nor B | |
| Hexetidine | 0.1% | neither A nor B | Coloured formula |
| | 0.05% | neither A nor B | (darkening with |
| | | | heat: 15 days at 50° C. |
| | | | and 2 months at 40° C.) |
| Chloroxylenol | 0.5% | emulsion | |
| | | impossible to | |
| | | manufacture | |
| | 0.1% | criteria A | Strong odour of cresol |
| | 0.01% | neither A nor B | even at 0.01% |
| | 0.001% | neither A nor B | |
| Chlorobutanol | 0.5% | criteria B | |
| hemihydrate | 0.1% | neither A nor B | |
| Phenoxy ethanol | 1% | criteria A | irritant |
| | 0.5% | neither A nor B | |
| Benzyl alcohol | 1% | criteria A | allergenic |
| | 0.5% | neither A nor B | |
| Boric acid | nt | | irritant |
| Sodium borate | nt | | irritant |
| Benzalkonium chloride | 0.25% | neither A nor B | allergenic |
| Triacetin | 1% | neither A nor B | |
| Phenylethyl alcohol | 0.5% | neither A nor B | |
| | 0.25% | neither A nor B | |
| Pentylene glycol | 5% | criteria B | (NB: fluid milk) |
| 1,2 hexanediol | 5% | criteria A | (NB: fluid milk) |
| | 1% | neither A nor B | |
| Potassium sorbate | 0.2% | neither A nor B | Slight sorbate |
| | | | odour |
| Sodium proprionate | 0.3% | neither A nor B | Pungent odour |
| 2-pyrrolidone | 5% | neither A nor B | NB: yellowing + |
| | | | odour as of 3 m at |
| | 7.5% | neither A nor B | 40° C. |
| Octyldodecanol | 5% | neither A nor B | |
| Methylene blue | 10 ppm | neither A nor B | colorant is |
| | | | instable à the |
| | | | lumière |
| Thesit (macrogol | 3% | neither A nor B | (NB: very fluid) |
| lauryl ether) | | | |
| Copper digluconate | 5% | nt | Unstable cream |
| | | | (dephasing at |
| | | | manufacture) |
| Zinc undecylenate | 0.5% | criteria B | Thick cream, |
| | | | graded appearance |
| Thymol | 0.5% | criteria A | Strong camphor |
| | | | odour |
| Anisic acid | 0.5% | nt | Pb of dispersion |
| | | | and |
| | | | recrystallisation |
| Bisabolol | 1% | neither A nor B | |
| Capryloyl glycine | 2.5% | nt | Granular cream |
| Undecylenol glycine | 2.5% | nt | Granular cream |
| Zinc oxide | 1% | neither A nor B | Granular, |
| several preparations) | | | consistent cream |
| Steareth-2 | 1% | neither A nor B | |
| | 0.5% | neither A nor B | |
| CHX digluconate | 0.1% | neither A nor B | CHX incriminated |
| Bitrex | 10 ppm | neither A nor B | |
| | (0.001%) | | |
| Sodium | 1% | criteria A | Release of formol! |
| hydroxymethylglycinate | | | |
| Refined sweet almond | 1% | neither A nor B | |
| oil | | | |
| Romacil fragrance | 0.5% | neither A nor B | |
| Cetylpyridinium | 0.1% | neither A nor B | |
| chloride | | | |
| Sodium acetate | 0.5% | neither A nor B | |
| trihydrate | | | |
| Hydroxyphenyl | 1% | nt | Dephasing |
| propamidobenzoic acid | | | |
| Polyquaternium 10 | 1% | neither A nor B | |
| Tryicaprylin (Miglyol | 5% | neither A nor B | |
| 808) | | | |
| Propyl gallate | 0.1% | neither A nor B | |
| Pentetic acid | 0.1% | neither A nor B | Slightly granular |
| | | | aspect |

Preservatives tested alone

TABLE 3

| | | Results of | |
|---|---|---|---|
| | | microbiological | |
| | | tests | |
| | Concentrations | (compliance | Comments/ |
| Preservatives tested | tested | with criteria) | other aspects |
| Zinc dioxide A/ | 1%/0.3% | neither A nor B | |
| sodium propionate | | | |
| Ethylhexyl glycerin | 0.5%/0.3% | criteria B | Liquid or fluid |
| (EHG)/caprylyl | | | white cream |
| glycol (=1,2- | | | |
| octanediol) | | | |
| | 0.25%/0.4% | criteria A | |
| | 0.2%/0.4% | criteria A | |
| Ethylhexyl glycerin | 0.15%/0.4% | criteria B | |
| (EHG)/pentylene | | | |
| glycol | | | |

### Preservatives Tested in Combination.

Results: most preservatives are not suitable in the body of formula under the targeted conditions, for various reasons: microbiological criteria, odour, organoleptic and physical/chemical criteria, release of formol, dephasing.

The diols tested (pentylene glycol, ethylhexyl glycerin, hexanediol, caprylyl glycol) do not prove to be the most compatible.

### Example 2: Tests of Physical/Chemical Properties

### 2.1) Test at Manufacture (T0)

After the tests of the different preservative systems, it turns out that very few products are suited in the particular conditions of a composition comprising an association of glycerol, Vaseline® petrolatum and liquid paraffin. In addition, certain efficient preservatives lead to a more or less severe drop in the consistency and/or in the viscosity of the emulsion. A fluidification of the formula is notably noted for pentylene glycol, ethylhexyl glycerin, hexanediol.

To study the possibility of compensating the effects of fluidification, the different preservatives are tested in the presence and in the absence of a gelling agent, firstly in a polyacrylamide type gelling agent.

To study the galenic properties, different parameters of the compositions were monitored. The physical/chemical characteristics of the compositions were measured by the methods described below.

The size of the globules being still less than 25 μm, this criterion is not represented in the table of results.

### Methods for Characterising the Compositions:

### Method for Measuring the Consistency

The measurement of consistency is carried out at room temperature (around 20° C.) in a 250 mL glass jar filled to 200 g. The measurement is performed using a TAXT+type texture analyser (Swantec). The consistency of the product is represented by the maximum resistance force of the probe in the product, which is directly proportional to the measured mass. The consistency value is expressed in g.

### Measurement Geometries:

250 g glass jar filled to 200 g
P25 steel cylinder

### Measurement Parameters:

Pre-speed: 4 mm/s
Speed: 8 mm/s
Post-speed: 8 mm/s
Displacement: 20 mm
Triggering threshold: 5 g

### Methodology for Preparing the Sample

Fill the container (glass jar) without introducing air into the product.

Tap the jar so as to pack down the cream and to obtain the smoothest surface possible.

Maintain the jar closed 24 before carrying out the measurement.

### Microscopic Characterisation

The analysis is carried out on an Olympus BX40 type optical microscope, equipped with a ×40 lens (×400 magnification)

### Methodology

Take up the cream using a spatula, form a test portion spread out between a slide and cover glass and record a photographic image. On the image calculate the total number of globules according to the following method:
determination of the number of globules on the horizontal line.
determination of the number of globules on the vertical line.

The total number of globules on the image corresponds to the multiplication of the two values.

### Measurement of the Viscosity

The viscosity is measured at room temperature (around 20° C.) using a Rheomat RM200 type viscometer. It is expressed in mPa·s.

### Method:

Spindle: Spindle 3
Pre-shear (s⁻¹): /
Spindle diameter: Cup 1
Shear (s⁻¹): 7.6 s⁻¹ for 1 min
Temperature: RT without regulation
Type of measurement: Step by step
Reading: at 60 seconds

### Formulation of the Tested Compositions, by Weight Compared to the Weight of the Composition:

around 15% of glycerol,
around 8% of Vaseline® petrolatum,
around 2% of liquid paraffin,
around 0.2 to 2% of trolamine,
around 1 to 5% of stearic acid,
around 2 to 10% of glycerol monostearate,
around 0.5 to 2.5% isopropyl myristate,
around 0.2 to 2% of dimethicone,
around 2 to 10% of polyethylene glycol 600,
components listed in the table,
made up to 100% with water.

The results at T0 of the physical characterisations of the screening tests are the following:

TABLE 4

| | | | | | Copolymer | | Consis- | Viscosity |
|---|---|---|---|---|---|---|---|---|
| | | | | | mixture of | | tency | T0 |
| no | BG | EHG | HX | PG | acrylamide* | pH | (g) T0 | (mPa · s) |
| 2.1 | | | 5% | | / | 8.5 | 48 | 5602 |
| 2.2 | | | 5% | | 0.5% | 8.5 | 183 | 8645 |
| 2.3 | | 0.5% | | | / | 8.7 | 59 | 4431 |
| 2.4 | | 0.5% | | | 0.5% | 8.7 | 99 | 11450 |
| 2.5 | | 0.5% | | | 1% | 8.6 | 275 | 30408 |
| 2.6 | | 1% | | | / | 8.7 | 67 | 5051 |
| 2.7 | | 1% | | | 0.5% | 8.6 | 106 | 12984 |
| 2.8 | | | | 5% | 0.5% | 8.5 | 60 | 7841 |
| 2.9 | | | | 5% | 1% | 8.5 | 181 | 18864 |
| 2.10 | | 1% | | | 0.6% | 8.6 | 121 | 12866 |
| 2.11 | | 0.5% | | 3% | 0.5% | 8.5 | 40 | 4691 |
| 2.12. | | 0.5% | 2% | | 0.5% | 8.4 | 34 | 4115 |
| 2.13 | 10% | | | | 0.5% | 8.6 | 172 | 23817 |

BG: (Butylene Glycol; = Butanediol)
HX: Hexanediol
EHG: Ethylhexyl glycerin
PG: Pentylene glycol

For these comparative examples, it turns out that the polyacrylamide type gelling agent (added at different concentrations) increases the consistency and the viscosity of the emulsions, thus improving the texture in the presence of preservatives that fluidify the formula. For a quantity of 1%, it is possible to observe an overcompensation in terms of consistency in a test.

The remainder of the evaluation consists in test of stability of the emulsions over time using certain selected combinations, as detailed below.

(*Copolymer mixture of acrylamide/sodium acryloyldimethyltaurate with isohexadecane and polysorbate, Sepineo®)

2.2) Further Characterisations of the Compositions: Stability after 3 Months at 40° C. and 25% RH

### Methods for Measuring Consistency/Viscosity as Described Above, at Room Temperature.

### Formulation of the Compositions Tested, by Weight Compared to the Weight of the Composition:

around 15% of glycerol,
around 8% of Vaseline® petrolatum,
around 2% of liquid paraffin,
around 0.2 to 2% of trolamine,
around 1 to 5% of stearic acid,
around 2 to 10% of glycerol monostearate,
0% or around 0.5 to 2.5% isopropyl myristate,
around 0.2 to 2% of dimethicone,
around 2 to 10% of polyethylene glycol 600,
components listed in the table,
made up to 100% with water.

TABLE 5

| | | | | | | Consistency | | Viscosity |
|---|---|---|---|---|---|---|---|---|
| | | | | Mixture | | (g) T3 | | T3 |
| | | | | copolymer | Consistency | months | Viscosity | months |
| no | EHG | HX | PG | acrylamide ** | (g) T0 | 40° C.* | T0 | 40° C. |
| 2.14 | | 5% | | 0.5% | 183 | 54 | 8645 | 2237 |
| | | | | | | (−70%) | | (−74%) |
| 2.15 | 0.5% | | | 0.5% | 99 | 86 | 11450 | 6016 |
| | | | | | | (−13%) | | (−47%) |
| 2.16 | 0.3% | | 0.4% | 0.5% | 56 | 60 | 7864 | 5440 |
| | | | | | | (+7%) | | (−31%) |
| 2.17 | 0.3% | | 0.4% | 0.7% | 77 | 125 | 12100 | 9584 |
| | | | | | | (+62%) | | (−21%) |
| 2.18 | 0.3% | | 0.4% | 0.85% | 99 | 169 | 15227 | 12905 |
| | | | | | | (+71%) | | (−15%) |

HX: Hexanediol
EHG: Ethylhexyl glycerin
PG: Pentylene glycol
*The consistency value T3 months is measured, at room temperature, after storage at 40° C. and 25% relative humidity.

For these comparative examples, it turns out that the effect on the texture of the formulas by the gelling agent of type copolymer polyacrylamide/the mixture acrylamide/sodium acryloyldimethyltaurate with isohexadecane and polysorbate is not satisfactory in the long term: it is not durable nor predictable due to an important maturation of the emulsion, with a large difference in the consistency (>45 g) and/or viscosity values between T0 and T3 months. In addition, the consistency and the viscosity are low for a formula.

A compensation of this phenomenon was sought by the association of another gelling agent, as detailed hereafter.

(**Copolymer mixture of acrylamide/sodium acryloyldimethyltaurate with isohexadecane and polysorbate, Sepineo®)

### Example 3.: Association of Gelling Agents & Preservatives

### Example 3.1: Gelling Agent Screening Test

Firstly, different combinations of gelling agents are tested, in the presence of the preservative hexanediol. The aim is to ensure that the targeted physical/chemical properties may be obtained with these combinations.

Characterisation methods & body of formulas equivalent to example 2.1.

TABLE 6

| | | | | | Mixture | | | |
|---|---|---|---|---|---|---|---|---|
| | | | Carbomer ® | | copolymer | | Consistency | Viscosity |
| no | HX | Xanthan | 980 | HPMC | acrylamide * | pH | (g) T0 | T0 |
| 3.1 | 5% | | 0.25% | | 0.6% | 7.4 | 189 | 27590 |
| 3.2 | 5% | | 0.10% | | 0.6% | 7.9 | 178 | 14736 |
| 3.3 | 5% | | 0.05% | | 0.6% | 8.0 | 176 | 11304 |
| 3.4 | 5% | | 0.05% | | 0.5% | 8.0 | 181 | 10923 |
| 3.5 | 5% | 0.20% | | | 0.6% | 7.9 | 161 | 9194 |
| 3.6 | 5% | | | 0.10% | 0.6% | 7.7 | 177 | 10406 |

HPMC: Hydroxypropyl methyl cellulose
HX: Hexanediol

Result: At T0, the consistency and viscosity values are acceptable for the combinations tested. In the remainder of the evaluation, other preservatives and the longer term stability are included in the test. This is detailed in example 3.2.

(*Copolymer mixture of acrylamide/sodium acryloyldimethyltaurate with isohexadecane and polysorbate; Sepineo®)

### Example 3.2: Test of Association of Gelling Agents & Preservatives

The aim is to test various combinations of preservatives and gelling agents to test their compatibility with the body of formula, and this is done for the date of manufacture and for the condition of storage for 3 months at elevated temperature (40° C.)

### Characterisation Methods Equivalent to Example 2.2

### Formulation of the Compositions Tested, by Weight Compared to the Weight of the Composition:

around 15% of glycerol,
around 8% of Vaseline® petrolatum,
around 2% of liquid paraffin,
around 0.2 to 2% of trolamine,
around 1 to 5% of stearic acid,
around 2 to 10% of glycerol monostearate,
around 0.5 to 2.5% of isopropyl myristate,
around 0.2 to 2% of dimethicone,
around 2 A 10% of polyethylene glycol 600,
components listed in the table,
made up to 100% with water.

TABLE 7

| | | | | | | | | | Consistency | | |
|---|---|---|---|---|---|---|---|---|---|---|---|
| | | | | | | Copolymer | | | (g) T3 | | Viscosity |
| | | | | | Carbomer ® | mixture of | | Consistency | months | Viscosity | T3 months |
| no | BG | EHG | PG | Xanthan | 980 | acrylamide ** | pH | (g) T0 | 40° C.* | T0 | 40° C. |
| 3.7 | 10% | 0.5% | | | 0.05% | 0.6% | 7.7 | 168 | 173 | 26938 | 17456 |
| | | | | | | | | | (+3%) | | (−35%) |
| 3.8 | | 0.5% | 3% | | 0.05% | 0.6% | 7.6 | 174 | 164 | 18218 | 10745 |
| | | | | | | | | | (−6%) | | (−41%) |
| 3.9 | | 0.5% | 5% | 0.20% | | 0.6% | 7.7 | 108 | 147 | 9041 | 6533 |
| | | | | | | | | | (+36%) | | (−28%) |
| 3.10 | 10% | 0.3% | | | 0.05% | 0.6% | 8.0 | 192 | 187 | 29200 | 20879 |
| | | | | | | | | | (−3%) | | (−28%) |
| 3.11 | | 0.3% | 0.4% | | 0.05% | 0.5% | 8.2 | 201 | 187 | 18210 | 12845 |
| | | | | | | | | | (−7%) | | (−29%) |
| 3.12 | | 0.5% | | | 0.025% | 0.5% | 8.2 | 142 | 146 | 13058 | 12442 |
| | | | | | | | | | (+3%) | | (−5%) |
| 3.13 | | 0.3% | 0.4% | | 0.025% | 0.5% | 8.1 | 125 | 160 | 12651 | 12224 |
| | | | | | | | | | (+28%) | | (−3%) |

BG: (Butylene Glycol; = Butanediol)
EHG: Ethylhexyl glycerin
PG: Pentylene glycol
*The consistency value at T3 months is measured, at room temperature, after storage at 40° C. and 25% relative humidity.

Further to the association of two types of gelling agents (of which a polyacrylamide type gelling agent, copolymer mixture of acrylamide/sodium acryloyldimethyltaurate with isohexadecane and polysorbate; Sepineo®), the compositions are very suitable in terms of consistency and viscosity for different combinations of preservatives, and this at T₀ as well as at T_{3 months} (measured after storage at 40° C. and 25% relative humidity).

(**Copolymer mixture of acrylamide/sodium acryloyldimethyltaurate with isohexadecane and polysorbate; Sepineo®)

1. Method preparing a stable composition in the form of an oil in water or water in oil emulsion comprising glycerol, vaseline, and liquid paraffin and at least one preservative different from parabens, wherein the method comprises:
combining
the emulsion with
at least two gelling agents of which one is a polyacrylamide type gelling agent and one is selected from the group consisting of xanthan gum, crosslinked polymers of acrylic acid of high molecular weight, hydroxyethyl cellulose, hydroxypropyl methylcellulose and mixtures thereof,
wherein the addition of at least two gelling agents provides stability of the composition over time.

2. The method according to claim 1, wherein the at least one preservative different from parabens is selected from the group consisting of hexanediol, ethylhexyl glycerin, pentylene glycol, butylene glycol, 1,2 octanediol (caprylyl glycol) and mixtures thereof.

3. The method according to claim 1, wherein the composition comprises
water at a concentration of between 30 and 80% by weight relative to the total weight of the composition,
glycerol at a concentration of between 10 and 20% by weight relative to the total weight of the composition,
petrolatum at a concentration of between 3 and 20% by weight relative to the total weight of the composition,
liquid paraffin at a concentration of between 0.5 and 5% by weight relative to the total weight of the composition.

4. The method according to claim 1, wherein the composition has a pH comprised between pH 7.1 and pH 8.9.

5. The method according to claim 1, wherein the composition has a consistency at manufacture comprised between 70 and 230 g.

6. The method according to claim 1, wherein the composition has a viscosity between 7000 mPa·s and 40000 mPa·s.

7. The method according to claim 1, wherein the composition has, after storage for 3 months at 40° C. and 25% relative humidity,
a consistency value comprised between 40 and 210 g at room temperature,
and a viscosity between 6000 mPa·s and 30000 mPa·s at room temperature.

8. The method according to claim 1, wherein said polyacrylamide type gelling agent is a copolymer mixture of acrylamide/sodium acryloyldimethyltaurate with isohexadecane and polysorbate.

9. Method for improving the stability over time of an oil in water or water in oil emulsion comprising glycerol, vaseline, and liquid paraffin and at least one preservative different from parabens, wherein the method comprises:
combining
the emulsion with
at least two gelling agents of which one is a polyacrylamide type gelling agent and one is selected from the group consisting of xanthan gum, crosslinked polymers of acrylic acid of high molecular weight, hydroxyethyl cellulose, hydroxypropyl methylcellulose and mixtures thereof,
wherein the addition of at least two gelling agents improves the stability of the composition over time.

10. The method according to claim 9, wherein the at least one preservative different from parabens is selected from the group consisting of hexanediol, ethylhexyl glycerin, pentylene glycol, butylene glycol, 1,2 octanediol (caprylyl glycol) and mixtures thereof.

11. The method according to claim 9, wherein the composition comprises
water at a concentration of between 30 and 80% by weight relative to the total weight of the composition,
glycerol at a concentration of between 10 and 20% by weight relative to the total weight of the composition,
petrolatum at a concentration of between 3 and 20% by weight relative to the total weight of the composition,
liquid paraffin at a concentration of between 0.5 and 5% by weight relative to the total weight of the composition.

12. The method according to claim 9, wherein the composition has a pH comprised between pH 7.1 and pH 8.9.

13. The method according to claim 9, wherein the composition has a consistency at manufacture comprised between 70 and 230 g.

14. The method according to claim 9, wherein the composition has a viscosity between 7000 mPa·s and 40000 mPa·s.

15. The method according to claim 9, wherein the composition has, after storage for 3 months at 40° C. and 25% relative humidity,
a consistency value comprised between 40 and 210 g at room temperature,
and a viscosity between 6000 mPa·s and 30000 mPa·s at room temperature.

16. The method according to claim 9, wherein said polyacrylamide type gelling agent is a copolymer mixture of acrylamide/sodium acryloyldimethyltaurate with isohexadecane and polysorbate.
